(19) United States
(12) Patent Application Publication
(10) Pub. No.: US 2011/0250664 A1
(43) Pub. Date: Oct. 13, 2011

(54) YEAST HAVING IMPROVED ETHANOL YIELD

(75) Inventor: Pingsheng Ma, Tianjin (CN)

(73) Assignee: Tianjin University, Tianjin (CN)

(21) Appl. No.: 13/083,365

(22) Filed: Apr. 8, 2011

Related U.S. Application Data
(60) Provisional application No. 61/325,147, filed on Apr. 16, 2010.
(60) Provisional application No. 61/321,963, filed on Apr. 8, 2010.

(51) Int. Cl. C12P 7/06; C12M 1/00; C12N 1/19

(52) U.S. Cl. 435/161; 435/254.2; 435/254.21; 435/254.23; 435/254.22; 435/289.1

(57) ABSTRACT

The present invention provides for a genetically-altered haploid yeast cell characterized by reduced activity or expression of FPS1, reduced activity or expression of glycerol-3-phosphate dehydrogenase-1 (GPD1), reduced activity or expression of glycerol-3-phosphate dehydrogenase-2 (GPD2), and increased expression of glutamate synthase (GLT1), wherein the reduced activity or expression, and the increased expression, is relative to expression or activity in a wildtype yeast strain, e.g., S. cervisiae strain S288C.

### CROSS-REFERENCE TO RELATED PATENT APPLICATIONS

The present patent application claims benefit of priority to U.S. Provisional Patent Application No. 61/321,963, filed Apr. 8, 2010, and U.S. Provisional Patent Application No. 61/325,147, filed Apr. 16, 2010, each of which are incorporated by reference.

### BACKGROUND OF THE INVENTION

It is generally recognized that the use of conventional petrochemical resources contributes to environmental effects that impact our global environment. It is also generally recognized that fossil fuels are limited. It is now clear that a new, sustainable technology that is based on renewable resources has to be developed. One way to do this is the production of ethanol and other energy carriers from feedstocks such as starch and cellulose. Therefore, efficient and robust microorganisms are needed for optimal and cost-efficient sugar-to-ethanol conversion.

### BRIEF SUMMARY OF THE INVENTION

The present invention provides a genetically-altered haploid yeast cell characterized by: a). reduced activity or expression of FPS1; b). reduced activity or expression of glycerol-3-phosphate dehydrogenase-1 (GPD1); c). reduced activity or expression of glycerol-3-phosphate dehydrogenase-2 (GPD2); and d). increased expression of glutamate synthase (GLT1), wherein the reduced activity or expression, and the increased expression, is relative to expression or activity in a wild type yeast strain, e.g., S. cervisiae strain S288C (ATCC Deposit No.: 204508).

In some embodiments, the haploid yeast cell has reduced expression of FPS1, GPD1, and GPD2. In some embodiments, the haploid yeast cell comprises a deletion in the coding region of FPS1, thereby preventing or reducing expression of an active FPS1 protein. In some embodiments, the haploid yeast cell comprises a deletion in the coding region of GPD2, thereby preventing or reducing expression of an active GPD2 protein. In some embodiments, the haploid yeast cell comprises a deletion in the coding region of GPD1, thereby preventing or reducing expression of an active GPD1 protein. In some embodiments, the haploid yeast cell comprises a deletion in the promoter region of FPS1, thereby preventing or reducing expression of an active FPS1 protein. In some embodiments, the haploid yeast cell comprises a deletion in the promoter region of GPD2, thereby preventing or reducing expression of an active GPD2 protein. In some embodiments, the haploid yeast cell comprises a deletion in the promoter region of GPD1, thereby preventing or reducing expression of an active GPD1 protein. In some embodiments, GLT1 expression in the haploid yeast cell of the present invention is increased due to a heterologous promoter operably linked to a GLT1 coding sequence, i.e., the heterologous promoter causes increased expression of GLT1. In some embodiments, the heterologous promoter comprises a yeast phosphoglycerate kinase (PGK) promoter.

In some embodiments, the haploid yeast cell comprises a). a deletion in the coding region of FPS1, thereby preventing or reducing expression of an active FPS1 protein; b). a deletion in the coding region of GPD2, thereby preventing or reducing expression of an active GPD2 protein; c). a deletion in the promoter region of GPD1, thereby preventing or reducing expression of an active GPD1 protein; and d). a heterologous promoter operably linked to a GLT1 coding sequence, wherein the heterologous promoter causes increased expression of GLT1. In some embodiments, the haploid yeast cell is genetically altered from Saccharomyces cerevisiae, S. bayanus, S. pastorianus, Pichia stipitis, or Candida shehatae. In some embodiments, the haploid yeast cell is a genetically-altered haploid yeast cell as deposited as ATCC Accession No. PTA-10766.

The present invention provides a method of generating ethanol from sugar comprising, contacting the yeast cell of the present invention to a solution comprising sugar under conditions such that the yeast cell converts the sugar to ethanol. The present invention further provides a system comprising a fermentation bioreactor and a haploid yeast cell of the invention.

### DEFINITIONS

The term “glycerol-3-phosphate dehydrogenase gene” and “GPD gene” are used herein to refer to any gene that encodes for a protein with glycerol-3-phosphate dehydrogenase activity. “Glycerol-3-phosphate dehydrogenase activity” refers to the ability of a protein to catalyze the reaction of dihydroxyacetone phosphate (DHAP) to glycerol-3-phosphate. Polypeptides having GPDH activity are assigned to Enzyme Classification (EC) 1.1.1.8 under the IUBMB Enzyme Nomenclature system. Representative GPDH nucleic acid and polypeptide sequences can be found, for example, in GenBank Accession Nos. NC_{—}003424.3; NC_{—}002951.2; NC_{—}009648.1; NT_{—}033779.4; NC_{—}000002.10; NC_{—}006322.1; NC_{—}003281.7; and NC_{—}003279.5. See, also, Baranowski, “α-Glycerophosphate dehydrogenase,” In: Boyer et al., (Eds.), The Enzymes, 2nd Ed., Vol. 7, Academic Press, New York, 1963, pp. 85-96. Representative GPD nucleic acid and polypeptide sequences can be found, for example, in GenBank Accession Nos. NC_{—}003424.3; NC_{—}002951.2; NC_{—}009648.1; NT_{—}033779.4; NC_{—}000002.10; NC_{—}006322.1; NC_{—}003281.7; and NC_{—}003279.5. For the purpose of the present invention, the term “GPD gene” are used herein to refer to any chromosomal DNA sequence that encodes for an enzyme that is at least 70 percent, preferably at least 80 percent and more preferably at least 90 percent identical to any of the polypeptide sequences identified in, e.g., GenBank Accession Nos. NC_{—}003424.3; NC_{—}002951.2; NC_{—}009648.1; NT_{—}033779.4; NC_{—}000002.10; NC_{—}006322.1; NC_{—}003281.7; and NC_{—}003279.5.

In S. cerevisiae, there are two genes designated GPD1 (e.g., Gene Identification No.: gi|6320181) and GPD2 (e.g., Gene Identification No.: gi|6324513) that each encode an active isoenzyme of NAD-dependent GPDH designated Gpdp. Despite the similar physical and catalytic properties of their gene products (Gpd1p and Gpd2p, respectively), the GPD1 and GPD2 genes are differentially regulated at the transcriptional level. Expression of GPD1 is induced by high osmolarity, whereas expression of GPD2 is induced under anaerobic conditions. Consistent with their transcriptional regulation, the enzyme encoded by GPD1 is predominantly responsible for adaptation of S. cerevisiae to high osmolarity, while that encoded by GPD2 is important for maintaining the cellular redox balance under anaerobic conditions.

The term “FPS1” refers to FPS1 gene encoding a glycerol permease designated Fps1p (e.g., Gene Identification No.: gi|6322985). The Fps1p channel is the mediator of the major part of glycerol passive diffusion (Oliveira et al., 2003). Glycerol transport polypeptides such as FPS1 are members of the major intrinsic protein (MIP) family of channel proteins. Among MIPs, two functionally distinct subgroups have been characterized; aquaporins, which allow specific water transfer, and glycerol channels, which are involved in glycerol transport and transport of small neutral solutes. Representative sequences of glycerol transport proteins (also known as glycerol channel polypeptides or facilitators) or variations thereof can be found, for example, in GenBank Accession Nos. NP_{—}013057; NC_{—}001144.4; NC_{—}007946.1; NC_{—}006155.1; NC_{—}010322.1; NC_{—}003143.1; NC_{—}002662.1; and NC_{—}000964.2. For the purpose of the present invention, the term “FPS1 gene” are used herein to refer to any chromosomal DNA sequence that encodes for an enzyme that is at least 70 percent, preferably at least 80 percent and more preferably at least 90 percent identical to any of the polypeptide sequences identified in, e.g., GenBank Accession Nos. NP_{—}013057; NC_{—}001144.4; NC_{—}007946.1; NC_{—}006155.1; NC_{—}010322.1; NC_{—}003143.1; NC_{—}002662.1; and NC_{—}000964.2.

The term “GLT1” are used herein to refer to any gene that encodes for a protein with glutamate synthase activity. Polypeptides having glutamate synthase activity are assigned EC 1.4.1.13 under the IUBMB Enzyme Nomenclature system. Representative GLT1 nucleic acid and polypeptide sequences can be found, for example, in GenBank Accession Nos. NC_{—}003071.4; NC_{—}001136.8; NC_{—}003424.3; NC_{—}007795.1; NC_{—}009077.1; NC_{—}009632.1; and NC_{—}010468.1. See, also, Miller & Stadtman, “Glutamate synthase from Escherichia coli. An iron-sulfide flavoprotein,” J. Biol. Chem., 247:7407-7419, 1972. For the purpose of the present invention, the term “GLT1 gene” are used herein to refer to any chromosomal DNA sequence that encodes for an enzyme that is at least 70 percent, preferably at least 80 percent and more preferably at least 90 percent identical to any of the polypeptide sequences identified in, e.g., GenBank Accession Nos. NC_{—}003071.4; NC_{—}001136.8; NC_{—}003424.3; NC_{—}007795.1; NC_{—}009077.1; NC_{—}009632.1; and NC_{—}010468.1.

“Reduced expression” or “reduced activity” as used herein means a decrease of at least 5%, 10%, 25%, 50%, 75%, or 100%, compared to a wild-type protein, polynucleotide, gene; or the activity and/or the concentration of the protein present before the polynucleotides or polypeptides are reduced. “Increase expression” as used herein means an increase of at least 5%, 10%, 25%, 50%, 75%, 100%, 200% or even more than 500%, compared to a wild-type protein, polynucleotide, gene; or the activity and/or the concentration of the protein present before the polynucleotides or polypeptides are reduced.

For the purposes of the present invention, the terms “wild-type yeast cell” or “wild-type yeast strain” refer to any yeast cell(s) containing no mutations in either the regulatory or coding regions of a GPD1, GPD2, FPS1 and GLT1 genes, and having no exogenously introduced GLT1, GPD1, GPD2, and FPS1 expression cassettes. Examples of wild type yeast cell include S. cervisiae strain S288C (ATCC Deposit No.: 204508).

A “promoter” is defined as an array of nucleic acid control sequences that direct transcription of an operably linked nucleic acid. As used herein, a “promoter” is a promoter that functions in yeast. Promoters include necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, optionally at least 65%, 70%, 75%, 80%, 85%, 90%, 95%, 98%, or 99% identity over a specified region (or the whole reference sequence when not specified)), when compared and aligned for maximum correspondence over a comparison window, or designated region as measured using one of the following sequence comparison algorithms or by manual alignment and visual inspection. Sequences having less than 100% identity but that have at least one of the above-specified percentages are said to be “substantially identical.” Optionally, the identity exists over a region that is at least about 50 nucleotides in length, or more preferably over a region that is 100 to 500 or 1000 or more nucleotides in length.

The term “similarity,” or “percent similarity,” in the context of two or more polypeptide sequences, refer to two or more sequences or subsequences that have a specified percentage of amino acid residues that are either the same or similar as defined in the 8 conservative amino acid substitutions defined above (i.e., 60%, optionally 65%, 70%, 75%, 80%, 85%, 90%, or 95% similar over a specified region), when compared and aligned for maximum correspondence over a comparison window, or designated region as measured using one of the following sequence comparison algorithms or by manual alignment and visual inspection. Sequences having less than 100% similarity but that have at least one of the specified percentages are said to be “substantially similar.” Optionally, this identity exists over a region that is at least about 50 amino acids in length, or more preferably over a region that is at least about 100 to 500 or 1000 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Examples of an algorithm that is suitable for determining percent sequence identity and sequence similarity include the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes or other nucleic acid sequences arranged to make a new functional nucleic acid, e.g., a promoter from one source and a coding region from another source.

An “expression cassette” is a nucleic acid construct, generated recombinantly or synthetically, with a series of specified nucleic acid elements that permit transcription of a particular nucleic acid in a host cell. The expression cassette can optionally be part of a plasmid, virus, or other nucleic acid fragment. Typically, the expression cassette includes a nucleic acid to be transcribed operably linked to a promoter.

### BRIEF DESCRIPTION OF THE DRAWINGS

FIG. 1: Alignment between the Mata fps1::URA3 PGKp-GLT1 gpd2::ura3 GPD1 promoter sequence (SEQ ID NO:1) and the wild type Saccharomyces cerevisiae GPD1 promoter sequence (SEQ ID NO:2). The last 60 nucleotides of the wild type Saccharomyces cerevisiae GPD1 promoter is absent in the Mata fps1::URA3 PGKp-GLT1 gpd2::ura3 GPD1 promoter sequence. The underlined bolded ATG represents the start codon. The −1, −61, and −607 positions of the wild type Saccharomyces cerevisiae GPD1 promoter sequence is shown.

### DETAILED DESCRIPTION

### Introduction

The present invention provides genetically-altered haploid yeast cells characterized by reduced activity or expression of FPS1, reduced activity or expression of glycerol-3-phosphate dehydrogenase-1 (GPD1), reduced activity or expression of glycerol-3-phosphate dehydrogenase-2 (GPD2), and increased expression of glutamate synthase (GLT1), as compared to wild-type yeast strains. Commercially available yeast cells for production of ethanol utilize diploid yeast cells and perform well. As discussed herein, the genetically-altered haploid yeast cells of the invention possess many remarkable properties and outperform commercially available diploid yeast cells.

It was surprisingly discovered in the present invention that the genetically-altered haploid yeast strains produce increased amount of ethanol and decreased amount of glycerol under conditions suitable for producing ethanol in yeast, as compared to other yeast strains, e.g., commercially available yeast strains. Despite that the glycerol biosynthesis pathway and the glycerol transport pathway are disrupted in the genetically-altered haploid yeast cells of the present invention, the growth of the yeast cells were not impaired. Even more unexpectedly, it was found that the genetically-altered haploid yeast strain produces decreased amount of iso-butanol, iso-amyl alcohol, and a-amyl alcohol under conditions suitable for producing ethanol in yeast, as compared to other yeast strains, e.g., commercially available yeast strains. Finally, the genetically-altered haploid yeast strain produces decreased amount of methanol, ethyl acetate, n-propanol, sec-butanol, n-butanol, acetic acid under conditions suitable for producing ethanol in yeast, as compared to other yeast strains.

### Methods of Reducing Protein Activity or Expression in a Cell

The genetically-altered haploid yeast cells of the present invention are characterized by reduced activity or expression of GPD1, GPD2 and FPS1. In some embodiments, the coding region and/or the promoter region of GPD1, GPD2, or FPS1 is deleted, thereby preventing expression of an active GPD1, GPD2, or FPS1 protein. Methods of deleting the coding/promoter region of a gene, such as the polymerase chain reaction (PCR) based gene replacement protocol (Guldener U, Heck S, Fielder T, Beinhauer J, Hegemann J H., Nucleic Acids Res 1996 Jul. 1; 24(13):2519-24, “A new efficient gene disruption cassette for repeated use in budding yeast.”) can be used to delete the coding region and/or the promoter region of GPD1, GPD2, or FPS1.

In some embodiments, the expression of GPD1, GPD2 or FPS1 is reduced by at least 50%, 60%, 70%, 80%, 90% compared to its expression in a wild type cell under its wild type promoter. It is of particular advantage to reduce expression by at least 80%, or at least 90%, and it is most preferred to reduce the expression by at least 95%, or at least 99%, compared to the expression of the particular gene in a wild type yeast cell, i.e. a yeast cell with a native promoter.

In some embodiments, the expression of GPD1, GPD2, and FPS1 can be reduced, resulting in a reduction of these proteins in the cell. This can be achieved by expressing GPD1, GPD2 or FPS1 by a weak promoter that is operably linked to these gene. A promoter is weak when the transcription rate of the gene is reduced to less than 50%, 20% 15%, 10%, 7% or 5% of the transcription rate of that gene expressed under the wild type promoter, i.e., the wild type promoter for GPD1, GPD2 or FPS1. Ways of measuring the strength of a promoter are known to a person of skill in the art, such as using a reporter gene like luciferase or green fluorescent protein (GFP), measuring the mRNA levels, e.g. using Northern blot or real-time reverse transcriptase PCR; on the protein level by Western blotting; or through measurements of the specific enzyme activity. In some embodiments, the weak promoter is a truncated promoter, e.g., a truncated GPD1 promoter (−607 to −61).

The reduction of the activity of GPD1, GPD2 or FPS1 protein can also be achieved by providing or expressing an antisense polynucleotides, siRNA, microRNA or other nucleic acids that prevent or impede the translation of GPD1, GPD2 or FPS1 mRNA into a protein.

### Methods of Increasing Protein Activity or Expression in a Cell

The genetically-altered haploid yeast cells of the present invention are characterized by increased activity or expression of GLT1. Since disrupting glycerol synthesis and/or transport of glycerol out of the cell alters the state of redox balance of a cell growing under anaerobic conditions due to an accumulation of NADH, the yeast can be engineered to effectively reoxidize the excess cytosolic NADH in the absence of glycerol synthesis. In some embodiments, excess NADH is effectively reoxidized by over-expressing a nucleic acid sequence encoding a glutamate synthase, which utilizes NADH as a co-factor in the conversion of glutamine to glutamate. It is also believed that polypeptides other than glutamate synthase can be over-expressed or disrupted provided that those polypeptides are involved, either directly or indirectly, in reactions that maintain the cellular redox balance (e.g., by reoxidizing NADH or NADPH). Such polypeptides include, for example, glutamine synthetase (GS) encoded by GLN1, NADP+-dependent glutamate dehydrogenases encoded by GDH1 and GDH3, or a NAD+-dependent glutamate dehydrogenase encoded by GDH2. Rather than over-expressing a nucleic acid, the encoded polypeptide (e.g., glutamate synthase, GS, NADP+-dependent glutamate dehydrogenase, or NAD+-dependent glutamate dehydrogenase) can be genetically-engineered to exhibit greater activity (compared to a wild type polypeptide) such that the chemical reaction that is facilitated by the genetically-engineered polypeptide takes place at a faster rate relative to the wild type polypeptide.

There are a number of ways in which a nucleic acid sequence encoding a polypeptide can be over-expressed. For example, the number of copies of a nucleic acid sequence can be increased; a nucleic acid sequence can be genetically engineered so as to be expressed under a different or stronger promoter and/or enhancer; the promoter and/or other regulatory elements of a nucleic acid sequence can be altered so as to direct high levels of expression (e.g., the binding strength of a promoter region for its transcriptional activators can be increased); the half-life of the transcribed mRNA can be increased; the degradation of the mRNA and/or polypeptide can be inhibited; and/or a nucleic acid sequence can be genetically modified as described herein such that the activity of the encoded polypeptide (e.g., rate of conversion, affinity for substrate) is increased. A nucleic acid sequence is considered to be over-expressed if the encoded polypeptide is present at an amount that is at least 20% higher (e.g. at least 25%, 30%, 40%, 50%, 60%, 70%, 80%, 90%, 95%, 99%, 100% higher or more) that will than the amount of polypeptide typically expressed from a corresponding nucleic acid that is not over-expressed. As used herein, “over-expression” also can refer to an increase in activity of a polypeptide (e.g., a polypeptide that has at least two-fold greater activity than a wild type polypeptide).

One or more copies of a nucleic acid sequence to be over-expressed can be present in a construct (also referred to as a vector), or one or more copies of a nucleic acid sequence to be over-expressed can be integrated into the yeast genome. Constructs suitable for over-expressing a nucleic acid are commercially available (e.g., expression vectors) or can be produced by recombinant DNA technology methods routine in the art. See, for example, Akada et al. (2002, Yeast, 19:17-28; and Mitchell et al. (1993, Yeast, 9:715-22). In addition, methods for stably integrating nucleic acid into the yeast genome are known and routine in the art. See, for example, Methods in Enzymology: Guide to Yeast Genetics and Molecular Biology, Vol. 194, 2004, Abelson et al., eds., Academic Press.

A construct containing a nucleic acid sequence can have elements necessary for expression operably linked to such a nucleic acid sequence, and further can include sequences such as those encoding a selectable marker (e.g., an antibiotic resistance gene), and/or those that can be used in purification of a polypeptide (e.g., 6×His tag). A construct also can include one or more origins of replication. Elements necessary for expression include nucleic acid sequences that direct and regulate expression of nucleic acid coding sequences. One example of an element necessary for expression is a promoter sequence.

Representative promoters include, without limitation, the promoter from the phosphoglycerate kinase (PGK) gene, the promoter from the triose phosphate isomerase (TPI1) gene, the promoter from the alcohol dehydrogenase (ADH1) gene, the promoter for translation elongation factor 1α (TEF), and the promoter for Glyceraldehyde-3-phosphate dehydrogenase (TDH3). Elements necessary for expression also can include intronic sequences, enhancer sequences, response elements, or inducible elements that modulate expression of a nucleic acid coding sequence.

### Genetically-Altered Haploid Yeast Cells

Under conditions suitable for producing ethanol in yeast, yeast cells described herein maintain normal cell growth and produce increased amount of ethanol, decreased amount of glycerol, and decreased amount of iso-butanol, iso-amyl alcohol, and a-amyl alcohol, as compared to other yeast strains, e.g., commercially available yeast strains. Any type of yeast can be genetically-engineered as described herein, including but not limited to, Saccharomyces cerevisiae, S. bayanus, S. pastorianus, Pichia stipitis, or Candida shehatae. Commercially available yeast includes, e.g., RED STAR® and ETHANOL RED® yeast (available from Fermentis/Lesaffre, USA), FALI (available from Fleischmann's Yeast, USA), SUPERSTART and THERMOSACC® fresh yeast (available from Ethanol Technology, Wis., USA), BIOFERM AFT and XR (available from NABC—North American Bioproducts Corporation, GA, USA), GERT STRAND (available from Gert Strand AB, Sweden), and FERMIOL (available from DSM Specialties).

The present invention further discloses a Saccharomyces cerevisiae cell line. The cells were deposited as ATCC Accession No. PTA-10766 pursuant to the Budapest Treaty at the American Type Culture Collection (ATCC), 10801 University Boulevard, Manassas, Va. 20110, on Apr. 6, 2010. Viability was tested on Apr. 14, 2010 and certified on Apr. 16, 2010.

The genetically-altered haploid yeast cells as described herein produce higher amounts of ethanol than yeast cells that do not have at least one genetic modifications (e.g., wild type yeast cells, or other commercially available yeast cells). The genetically-altered haploid yeast cells described herein can produce ethanol at levels that are increased by up to about 3% or more, 4% or more, 5% or more, 6% or more, 7% or more, 8% or more, 9% or more, 10% or more, 11% or more, 12% or more, 13% or more, 14% or more, 15% or more compared to other yeast cells. In some embodiments, the other yeast cells are S288C cells. In some embodiments, the other yeast cells are other commercially available yeast cells, e.g., BIOFERM XR or ETHANOL RED®. In some embodiments, the other yeast cells are cells of a/αfps1::URA3 PGKp-GLT1 gpd2::ura3 strain.

Further, the genetically-altered haploid yeast cells described herein can produce at least 20% (e.g., at least 25%, 30%, 35%, 40%, 45%, 50%, 60%, or more) less glycerol compared to other yeast cells. In some embodiments, the other yeast cells are other commercially available yeast cells, e.g., BIOFERM XR or ETHANOL RED®.

In a fermentation process, yeast can take other alternative pathways such as the iso-Butanol pathway, a-Amyl alcohol pathway, and iso-Amyl alcohol pathway. The genetically-altered haploid yeast cells described herein can produce at least 10% less (e.g., at least 15%, 20%, 25%, 30%, 35%, 40%, 45%, 50%, 55%, 60%, 70%, 80%, 90% less) iso-Butanol, a-Amyl alcohol, or iso-Amyl alcohol compared to other yeast cells. Further, the genetically-altered haploid yeast cells as described herein can produce at least 10% less (e.g., at least 15%, 20%, 25%, 30%, 35%, 40%, 45%, 50%, 55%, 60%, 70%, 80%, 90% less) methanol or ethyl acetate compared to other yeast cells. In some embodiments, the other yeast cells are other commercially available yeast cells, e.g., BIOFERM XR or ETHANOL RED®. In some embodiments, the other yeast cells are cells of a/αfps1::URA3 PGKp-GLT1 gpd2::ura3 strain.

Further, in some embodiments, the genetically-altered haploid yeast cells as described herein can produce at least 10% less (e.g., at least 15%, 20%, 25%, 30%, 35%, 40%, 45%, 50%, 55%, 60%, 70%, 80%, 90% less) n-propanol or n-butanol compared to other yeast cells, e.g., a/a fps1:: URA3 PGKp-GLT1 gpd2::ura3 yeast cells.

In some embodiments, the genetically-altered haploid yeast cells as described herein can produce at least 10% less (e.g., at least 15%, 20%, 25%, 30%, 35%, 40%, 45%, 50%, 55%, 60%, 70%, 80%, 90% less) acetic acid compared to other yeast cells, e.g., BIOFERM XR or ETHANOL RED®.

### Method of Using the Yeast Cells to Produce Ethanol

The present invention further provides a method of generating ethanol from sugar by contacting the yeast cells of the present invention to a solution comprising sugar under conditions such that the yeast cells converts the sugar to ethanol. For example, sugars are contacted with the genetically-altered haploid yeast cells of the invention under appropriate fermentation conditions, e.g., anaerobic conditions. The genetically-engineered yeast cells described herein can be used to convert sugar to ethanol during the fermentation of any type of biomass. As used herein, biomass includes, but is not limited to, crops such as starch crops (e.g., corn, wheat, rice or barley), sugar crops (e.g., sugarcane, energy cane or sugarbeet), forage crops (e.g., grasses, alfalfa, or clover), and oilseed crops (e.g., soybean, sunflower, or safflower); wood products such as trees, shrubs, and wood residues (e.g., sawdust, bark or the like from forest clearings and mills); waste products such as municipal solid waste (MSW; e.g., paper, food and yard wastes or wood), and process waste; and aquatic plants such as algae, water weed, water hyacinth, or reed and rushes.

Once sugars are converted into ethanol by the methods and processes of the invention, the ethanol can be collected, e.g., from a fermentation culture using standard distillation methods.

### EXAMPLES

The following examples are offered to illustrate, but not to limit the claimed invention.

### Example 1

### Construction of a/αfps1::URA3 PGKp-GLT1 gpd2::ura3

The a/αfps1::URA3 PGKp-GLT1 gpd2::ura3 strain used in the present invention was derived from a YC-DM yeast strain.

Construction of a selectable marker-recoverable gene knockout cassette: For multi-round gene manipulation, we made a URA3 based gene knockout cassette, in which, the URA3 gene can be used repeatedly as a selectable marker for multiple gene manipulation. To this end, plasmid pUC18-RYUR was constructed. First, a 435 bp DNA fragment corresponding to nucleotide sequence 4165652 bp to 4166066 of B. subtilis 168 genome were PCR amplified with primers Rep1-U (5′-GGG CCC GGA TCC GAG CAG CAT AAA CGA CTG CT-3′; SEQ ID NO:3) and Rep1-D (5′-GGG CCC TCT AGA ACG CTC AAT GTT GTT CAT GA-3′; SEQ ID NO:4) flanked by the restriction sites BamHI and XbaI, respectively. The resulting PCR product was digested by BamHI and XbaI and then ligated with the same enzyme pair digested pUC18, resulting in plasmid pUC18-R.

Second, the yeast URA3 gene was PCR amplified from YEplac195 with primers URA3-U (5′-GGG CCC TCT AGA GTA GTC TAG TAC CTC CTG TG-3′; SEQ ID NO:5), corresponding to the vector sequence 1940 to 1959 flanked by restriction site XbaI and URA3-D (5′-GGG CCC GTC GAC GAA AAG TGC CAC CTG ACG TC-3′; SEQ ID NO:6) corresponding to the vector sequence 3323 to 3304 flanked by restriction site SaiI, respectively. The resulting PCR product was digested by XbaI and SaiI and then ligated with the same enzyme pair digested pUC18-R, resulting in plasmid pUC18-RYU.

Finally, the exact same DNA sequence of B. subtilis 168 genome as described above was PCR amplified with primers Rep2-U (5′-GGG CCC GTC GAC GAG CAG CAT AAA CGA CTG CT-3′; SEQ ID NO:7) and Rep2-D (5′-GGG CCC CTG CAG ACG CTC AAT GTT GTT CAT GA-3′; SEQ ID NO:8) flanked by restriction sites SaiI and PstI, respectively. The resulting PCR fragment was digested by SaiI and PstI and then ligated with the same enzyme pair digested plasmid pUC18-RYU, creating plasmid pUC18-RYUR.

Deletion of FPS1: To delete FPS1, plasmid pUC18-RYUR was PCR amplified with primers KFPS1-U (5′-TCA ACA AAG TAT AAC GCC TAT TGT CCC AAT AAG CGT CGG TAC GAC GTT GTA AAA CGA CG-3′; SEQ ID NO:9) and KFPS1-D (5′-CAT CAT GTA TAG TAG GTG ACC AGG CTG AGT TCA TGT CAA CGG AAA CAG CTA TGA CCA TG-3′; SEQ ID NO:10). KFPS1-U contains, at its 3′ portion, sequences corresponding to pUC18 sequences 371 to 389 and, at its 5′ portion, sequences corresponding to positions −100 to −61 with respect to the ATG start codon of the FPS1 gene; KFPS1-D contains, at its 3′ portion, sequences corresponding to pUC18 sequences 479 to 461 and, at its 5′ portion, sequences corresponding to positions 2250 to 2211 with respect to the ATG start codon of the FPS1 gene. This PCR product was then used to transform yeast. Transformants were isolated on minimal medium lacking uracil and checked by diagnostic PCR for the correct integration of the RYUR cassette. The isolates, in which the targeted gene deletion had occurred, were subjected onto FOA plates to select for loop-out of the URA3 gene through homologous recombination between the repeat sequences flanking the URA3 gene in the deletion cassette.

Deletion of GPD2: For deletion of GPD2, plasmid pUC18-RYUR was PCR amplified with primers KGPD2-U (5′-CTC TTT CCC TTT CCT TTT CCT TCG CTC CCC TTC CTT ATC AAC GAC GTT GTA AAA CGA CG-3′; SEQ ID NO:11) and KGPD2-D (5′-GCA ACA GGA AAG ATC AGA GGG GGA GGG GGG GGG AGA GTG TGG AAA CAG CTA TGA CCA TG-3′; SEQ ID NO:12). KGPD2-U contains, at its 3′ portion, sequences corresponding to pUC18 sequences 371 to 389 and, at its 5′ portion, sequences corresponding to positions −40 to −1 with respect to the ATG start codon of the GPD2 gene; KGPD2-D contains, at its 3′ portion, sequences corresponding to pUC18 sequences 479 to 461 and, at its 5′ portion, sequences corresponding to positions 1363 to 1324 with respect to the ATG start codon of the GPD2 gene. This PCR product was then used to create GPD2 deletion strain as described above for deletion of FPS1.

Over-expression of GLT1: For GLT1 over-expression, plasmid YIplac211-Ppgk1-GLT1 that harbors 5′ portion of the GLT10RF fused to the PGK1 promoter and, upstream of the PGK1 promoter, a DNA fragment corresponding to positions 18 to −920 with respect to the ATG start codon of the GLT1 gene, was constructed as follows: (1) the first 1390 bp GLT10RF was PCR amplified with primers GLT1-U (5′-GGG CCC GTC GAC ATG CCA GTG TTG AAA TCA GA-3′; SEQ ID NO:13) corresponding to position 1 to 20 with respect to the ATG start codon of the GLT1 gene, flanked by the restriction site SaiI, and GLT1-D (5′-GGG CCC CTG CAG TTT TAG TAT CGA CCA TTT CA-3′; SEQ ID NO:14) corresponding to position 1390 to 1371 with respect to the ATG start codon of the GLT1 gene flanked by the restriction site PstI, respectively. The resulting PCR product was digested by SaiI and PstI and then ligated with the same enzyme pair digested YIplac211, resulting in plasmid YIplac211-GLTIt; (2) Primers GLT1prom-U (5′-GGG CCC GGT ACC TTT CTG AGC ACT GTC AGG AG-3′; SEQ ID NO:15) corresponding to position −920 to −901 with respect to the ATG start codon of the GLT1 gene flanked by the restriction site KpnI, and GLT1prom-D (5′-GGG CCC GGA TCC TGA TTT CAA CAC TGG CAT GC-3′; SEQ ID NO:16) corresponding to position 18 to −2 with respect to the ATG start codon of the GLT1 gene flanked by the restriction site BamHI were used to amplify a DNA fragment upstream of the GLT10RF. This PCR product was digested by KpnI and BamHI and then ligated with the same enzyme pair digested YIplac211-GLTIt, creating plasmid YIplac211-GLTIp-GLTIt. (3) Primers PGK1prom-U (5′-GGG CCC GGA TCC AGG CAT TTG CAA GAA TTA CTC-3′; SEQ ID NO:17) corresponding to position −701 to −721 with respect to the ATG start codon of the PGK1 gene flanked by the restriction site BamHI, and PGK1prom-D (5′-GGG CCC GTC GAC TGT TTT ATA TTT GTT GTA AAA AGT AG-3′; SEQ ID NO:18) corresponding to position −1 to −26 with respect to the ATG start codon of the PGK1 gene flanked by the restriction site SaiI were used to amplify a DNA fragment upstream of the PGK10RF that contains the promoter of the gene. This PCR product was digested by BamHI and SaiI and ligated with same enzyme pair digested YIplac211-GLT1p-GLT1.

To replace GLT1 promoter with the PGK1 promoter in the genome, YIplac211-Ppgk1-GLT1 was digested by BglI and the linearized plasmid was used for yeast transformation. Isolation and verification of the transformants and subsequent loop-out of the vector sequence, including the URA3 gene, were performed essentially as described above.

### Example 2

### Construction of Mata fps1::URA3 PGKp-GLT1 gpd2::ura3 PT₆₀-GPD1

The a Mata fps1::URA3 PGKp-GLT1 gpd2::ura3 PT₆₀-GPD1 strain used in the present invention was derived from a YC-DM yeast strain.

Mata fps1:: URA3 PGKp-GLT1 gpd2::ura3 PT₆₀-GPD1 was constructed using the same procedures outlined in Example 1 for the construction of a/αfps1::URA3 PGKp-GLT1 gpd2::ura3, except that GPD1 was not deleted in the construction of Mata fps1::URA3 PGKp-GLT1 gpd2::ura3 PT₆₀-GPD1. Instead, in the construction of Mata fps1:: URA3 PGKp-GLT1 gpd2::ura3 PT₆₀-GPD1, the wild type GPD1 promoter was replaced with a truncated GPD1 promoter (−607 to −61). The replacement of the wild type GPD1 with PT₆₀-GPD1 is discussed herein.

Replacement of the wild type GPD1 with PT₆₀-GPD1: First, the truncated GPD1 promoter (−607 to −61) was amplified from yeast genomic DNA using primer pairs pGPD1-U (5′ gggcccGAATTCcacttctacttctacatcg3′; SEQ ID NO:19), containing the restriction site for EcoRI and nucleotides −608 to −588 of GPD1, and pGPD1-D (5′ gggcccGGATCCttgatgtcttatgtaggagag3′; SEQ ID NO:20), containing the restriction site for BamHI and nucleotides −61 to −81 of GPD1. This PCR product was digested with EcoRI and BamHI and ligated with the same enzyme pair digested YIplac211, creating plasmid pJ130ᵢₙₜₑᵣ.

Second, the coding sequence plus the terminator sequence of GPD1 (+1 to +1461) was amplified from yeast genomic DNA using primer pairs GPD1-U (5′ gggcccGGATCCatgtctgctgctgctgatag3′; SEQ ID NO:21), containing the restriction site for BamHI and nucleotides +1 to +20 of GPD1, and GPD1-D (5′ gggcccAAGCTTtgagtggtgttgtaaccacc3′; SEQ ID NO:22), containing the restriction site for HindIII and nucleotides +1461 to +1442 of GPD1. This PCR product was digested with BamHI and HindIII and ligated with the same enzyme pair digested plasmid pJ130ᵢₙₜₑᵣ, generating plasmid pJ130.

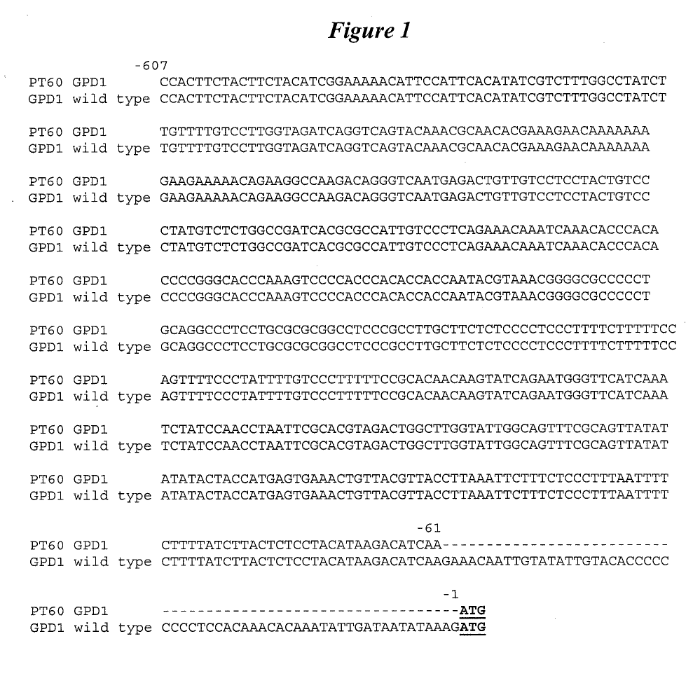

Third, plasmid pJ130 was linearized by SnaBI and transformed into yeast genome. Through a popin-popout procedure, the wild type GPD1 gene was replaced by the PT₆₀-GPD1 allele. This result was verified by analytical PCR (FIG. 1).

Sequencing genetically modified region of Mata fps1::URA3 PGKp-GLT1 gpd2::ura3 PT₆₀-GPD1:

The sequence of PT₆₀-GPD1 locus was determined as the following:

(SEQ ID NO: 23)
CCACTTCTACTTCTACATCGGAAAAACATTCCATTCACATATCGTCTTTG
GCCTATCTTGTTTTGTCCTTGGTAGATCAGGTCAGTACAAACGCAACACG
AAAGAACAAAAAAAGAAGAAAAACAGAAGGCCAAGACAGGGTCAATGAGA
CTGTTGTCCTCCTACTGTCCCTATGTCTCTGGCCGATCACGCGCCATTGT
CCCTCAGAAACAAATCAAACACCCACACCCCGGGCACCCAAAGTCCCCAC
CCACACCACCAATACGTAAACGGGGCGCCCCCTGCAGGCCCTCCTGCGCG
CGGCCTCCCGCCTTGCTTCTCTCCCCTCCCTTTTCTTTTTCCAGTTTTCC
CTATTTTGTCCCTTTTTCCGCACAACAAGTATCAGAATGGGTTCATCAAA
TCTATCCAACCTAATTCGCACGTAGACTGGCTTGGTATTGGCAGTTTCGC
AGTTATATATATACTACCATGAGTGAAACTGTTACGTTACCTTAAATTCT
TTCTCCCTTTAATTTTCTTTTATCTTACTCTCCTACATAAGACATCAAGG
ATCCATGTCTGCTGCTGCTGATAGATTAAACTTAACTTCCGGCCACTTGA
ATGCTGGTAGAAAGAGAAGTTCCTCTTCTGTT

The sequence underlined is the truncated GPD1 promoter (−607 to −61). The sequence in italic is the 5′ portion of the GPD10RF immediately after the start codon ATG. The GGATCC sequence right before the ATG start codon is the BamHI site used for fusion of GPD1 ORF with the truncated promoter.

The sequence of the PGKp-GLT1 locus was determined as the following:

(SEQ ID NO: 24)
CGTCTCCACATCATAGGAAGATAGGAAATTGCTATCTCAGTCCTATACTA
CGCAGACGGATACTCTCAGTTGCTCTTTCTTCCCCTTCTTTTAGCTCATT
GAGGTAGTGATTAACGTTTAACTTATTTATTTATTTTTCTGCTTCAGTTT
TTTTTTATTTATTTTTGTCTTTCTACTCTCTCTTTTTTCTTAATCTATTT
GCCATTTATTTATTTTGAAGAACTAGAAAAAGAATTAGAAAAGAAAGCAT
GCCAGTGTTGAAATCAGGATCCAGGCATTTGCAAGAATTACTCGTGAGTA
AGGAAAGAGTGAGGACTATCGCATACCTGCATTTAAAGATGCCGATTTGG
GCGCGAATCCTTTATTTTGGCTTCACCCTCATACTATTATCAGGGCCAGA
AAAAGGAAGTGTTTCCCTCCTTCTTGAATTGATGTTACCCTCATAAAGCA
CGTGGCCTCTTATCGAGAAAGAAATTACCGTCGCTCGTGATTTGTTTGCA
AAAAGAACAAAACTGAAAAAACCCAGACACGCTCGACTTCCTGTCTTCCT
ATTGATTGCAGCTTCCAATTTCGTCACACAACAAGGTCCTAGCGACGGCT
CACAGGTTTTGTAACAAGCAATCGAAGGTTCTGGAATGGCGGGAAAGGGT
TTAGTACCACATGCTATGATGCCCACTGTGATCTCCAGAGCAAAGTTCGT
TCGATCGTACTGTTACTCTCTCTCTTTCAAACAGAATTGTCCGAATCGTG
TGACAACAACAGCCTGTTCCCACACACTCTTTTCTTCTAACCAAGGGGGT
GGTTTAGTTTAGTAGAACCTCGTGAAACTTACATTTACATATATATAAAC
TTGCATAAATTGGTCAATGCAAGAAATACATATTTGGTCTTTTCTAATTC
GTAGTTTTTCAAGTTCTTAGATGCTTTCTTTTTCTCTTTTTTACAGATCA
TCAAGGAAGTAATTATCTACTTTTTACAACAAATATAAAACAGTCGACAT
GCCAGTGTTGAAATCAGACAATTTCGATCCATTGGAAGAAGCTTACGAAG
GTGGGACAATTCAAAACTATAACGATGAACACCATCTTCATAAATCTTGG
GCAAATGTGATTCCGGACAAACGAGGACTTTACGACCCTGATTATGA

The double-underlined sequence is the GLT1 promoter and the first 18 nucleotides of the GLT1 ORF sequence (−248 to +18). The sequence underlined is the PGK1 promoter (−721 to −1) and the sequence in italic is the 5′ portion of GLT1 ORF immediately after the ATG start codon. The underlined italic GGATCC (BamHI site) and GTCGAC (SalI site) sequences were introduced by PCR for cloning.

The sequence of the gpd2::ura3 locus was determined as the following:

(SEQ ID NO: 25)
TTTTTTTTATATATTAATTTTTAAGTTTATGTATTTTGGTAGATTCAATT
CTCTTTCCCTTTCCTTTTCCTTCGCTCCCCTTCCTTATCAATGCTAAGAG
ATAGTGATGATATTTCATAAATAATGTAATTCTATATATGTTAATTACCT
TTTTTGCGAGGCATATTTATGGTGAAGAATAAGTTTTGACCATCAAAGAA
GGTTAATGTGGCTGTGGTTTCAGGGTCCATAAAGCTTTTCAATTCATCAT
TTTTTTTTATTCTTTTTTTTGATTCCGGTTTCCTTGAAATTTTTTTGATT
CGGAAATCTCCGAACAGAAGGAAGAACGAAGGAAGGAGCACAGACTTAGA
TTGGTATATATACGCATATGTAGTGTTGAAGAAACATGAAATTGCCCAGT
ATTCTTAACCCAACTGCACAGAACAAAAACCTGCAGGAAACGAAGATAAA
TCATGTCGAAAGCTACATATAAGGAACGTGCTGCTACTCATCCTAGTCCT
GTTGCTGCCAAGCTATTTAATATCATGCACGAAAAGCAAACAAACTTGTG
TGCTTCATTGGATGTTCGTACCACCAAGGAATTACTGGAGTTAGTTGAAG
CATTAGGTCCCAAAATTTGTTTACTAAAAACACATGTGGATATCTTGACT
GATTTTTCCATGGAGGGCACAGTTAAGCCGCTAAAGGCATTATCCGCCAA
GTACAATTTTTTACTCTTCGAAGACAGAAAATTTGCTGACATTGGTAATA
CAGTCAAATTACAGTACTCTGCGGGTGTATACAGAATAGCAGAATGGGCA
GACATTACGAATGCACACGGTGTGGTGGGCCCAGGTATTGTTAGCGGTTT
GAAGCAGGCGGCAGAAGAAGTAACAAAGGAACCTAGAGGCCTTTTGATGT
TAGCAGAATTGTCATGCAAGGGCTCCCTAGCTACTGGAGAATATACTAAG
GGTACTGTTGACATTGCGAAGAGCGACAAAGATTTTGTTATCGGCTTTAT
TGCTCAAAGAGACATGGGTGGAAGAGATGAAGGTTACGATTGGTTGATTA
TGACACCCGGTGTGGGTTTAGATGACAAGGGAGACGCATTGGGTCAACAG
TATAGAACCGTGGATGAAGTGGTCTCTACAGGATCTGACATTATTATTGT
TGAAAGAGGGCTATTTGCAAAGGGAAGGGATGCTAAGGTAGAGGGTGAAC
GTTACAGAAAAGCAGGCTGGGAAGCATATTTGAGAAGATGCGGCCAGCAA
AACTAAAAAACTGTATTATAAGTAAATGCATGTATACTAAACTCACAAAT
TAGAGCTTCAATTTAATTATATCAGTTATTACCCGGGAATCTCGGTCGTA
ATGATTTTTATAATGACGAAAAAAAAAAATTGGAAAGAAAAAGCTTCATG
GCCTTTATAAAAAGGAACTATCCAATACCTCACACTCTCCCCCCCCCTCC
CCCTCTGATCTTTCCTGTTGCCTCTTTTTCCCCCAACCAATTTATCATTA
TACACAAGTTCTACAACTACTACTAGTAATATTACTACAGTTATTATAAT
TTTCTATTCTCTTTTTCTTTAAGAATCTATCATTAACGTTAAT

The underlined sequence is GPD2 promoter (−90 to −1) and The double-underlined sequence is GPD2 terminator (+1324 to +1485). The sequence in italic is the URA3 gene (−364 to +980). The underlined italic ATG is the start codon of the URA3 gene. The underlined italic TAA is the stop codon of URA3.

The sequence of the fps1::URA locus was determined as the following:

(SEQ ID NO: 26)
TTTTGCGGAAAGATAAAACAAGATATATTGCACTTTTTCCACCAAGAAAA
ACAGGAAGTGGATTAAAAAATCAACAAAGTATAACGCCTATTGTCCCAAT
AAGCGTCGGTTGTTCTTCTTTATTATTTTACCAAGTACGCTCGAGGGTAC
ATTCTAATGCATTAAAAGACATGCTAAGAGATAGTGATGATATTTCATAA
ATAATGTAATTCTATATATGTTAATTACCTTTTTTGCGAGGCATATTTAT
GGTGAAGAATAAGTTTTGACCATCAAAGAAGGTTAATGTGGCTGTGGTTT
CAGGGTCCATAAAGCTTTTCAATTCATCATTTTTTTTTTATTCTTTTTTT
TGATTCCGGTTTCCTTGAAATTTTTTTGATTCGGTAATCTCCGAACAGAA
GGAAGAACGAAGGAAGGAGCACAGACTTAGATTGGTATATATACGCATAT
GTAGTGTTGAAGAAACATGAAATTGCCCAGTATTCTTAACCCAACTGCAC
AGAACAAAAACCTGCAGGAAACGAAGATAAATCATGTCGAAAGCTACATA
TAAGGAACGTGCTGCTACTCATCCTAGTCCTGTTGCTGCCAAGCTATTTA
ATATCATGCACGAAAAGCAAACAAACTTGTGTGCTTCATTGGATGTTCGT
ACCACCAAGGAATTACTGGAGTTAGTTGAAGCATTAGGTCCCAAAATTTG
TTTACTAAAAACACATGTGGATATCTTGACTGATTTTTCCATGGAGGGCA
CAGTTAAGCCGCTAAAGGCATTATCCGCCAAGTACAATTTTTTACTCTTC
GAAGACAGAAAATTTGCCGACATTGGTAATACAGTCAAATTGCAGTACTC
TGCGGGTGTATACAGAATAGCAGAATGGGCAGACATTACGAATGCACACG
GTGTGGTGGGCCCAGGTATTGTTAGCGGTTTGAAGCAGGCGGCAGAAGAA
GTAACAAAGGAACCTAGAGGCCTTTTGATGTTAGCAGAATTGTCATGCAA
GGGCTCCCTAGCTACTGGAGAATATACTAAGGGTACTGTTGACATTGCGA
AGAGCGACAAAGATTTTGTTATCGGCTTTATTGCTCAAAGAGACATGGGT
GGAAGAGATGAAGGTTACGATTGGTTGATTATGACACCCGGTGTGGGTTT
AGATGACAAGGGAGACGCATTGGGTCAACAGTATAGAACCGTGGATGATG
TGGTCTCTACAGGATCTGACATTATTATTGTTGGAAGAGGACTATTTGCA
AAGGGAAGGGATGCTAAGGTAGAGGGTGAACGTTACAGAAAAGCAGGCTG
GGAAGCATATTTGAGAAGATGCGGCCAGCAAAACTAAAAAACTGTATTAT
AAGTAAATGCATGTATACTAAACTCACAAATTAGAGCTTCAATTTAATTA
TATCAATTATTACCCGGGAATCTCGGTCGTAATGATTTCTATAATGACGA
AAAAAAAAAAATTGGAAAGAAAAAGCTTCATGGCCTTTATAAAAAGGAAC
ATCCAATACCTTGAGAAAACAGACAAGAAAAAGAAACAAATAATATAGAC
TGATAGAAAAAAATACCGCTTACTACCGCCGGTATAATATATATATATAT
ATTTACATAGATGATTGCATAGTGTTTTAAAAAGCTTTCCTAGGTTAAGC
TATGAATCTTCATAACCTAACCAACTAAATATGAAAATACTGACCCATCG
TCTTAAGTAAGTTGACATGAACTCAGCCTGGTCACCTACTATACATGATG
TATCGCATGGATGGAAAGAATACCAAACGCTACCTTCCAGGTTAATGATA
GTATCCAAACCTAGTTGGAATTTGCCTTGAACATCAAGCAGCGATTCGAT
ATCAGTTGGGAGCATCAATTTGGTCATTGGAATACCATCTATGCTTTTCT
CCTCCCATATTCTCAAAAGTAGTAAGGGCTCGTTATATACTTTTGAATAT
GTAAGATATAATTCTATATGATTTAGTAATTTATTTTCTATACGCTCAGT
ATTTTTCTGCAGTTGTCGAGTAGGTATTAAACGCAGAAGAAGTCCATCCT
TCTCATCATTCAAATGAACATCTTGGCAAA

The underlined sequence is FPS1 promoter (−171 to −1) and the double-underlined sequence is FPS1 terminator (+2008 to +2580). The sequence in italic is the URA3 gene (−364 to +980). The underlined italic ATG is the start codon of the URA3 gene and the underlined italic TAA is the stop codon of the URA3 gene.

### Example 3

### Ethanol and Glycerol Yields of Mata fps1::URA3 PGKp-GLT1 gpd2::ura3 PT₆₀-GPD1 as Compared to Other Commercially Available Yeast Cells and a/αfps1::URA3 PGKp-GLT1 gpd2::ura3

Corn mash was prepared at ˜32.6% sugars with 31.13% solids as measured by a microwave moisture analyzer. Standard preparation of the corn mash was completed using freshly ground corn and Alpha-amylase enzyme. The approximate Brix of the material was 26.0 and the starting pH of the corn mash was 4.8. A total of 500 ppm of Nitrogen (270 ppm from Ammonia and 230 ppm N from urea) along with 5 ppm of each of the following antibiotics, Allpen and Lactoside V were put into the corn mash prior to inoculation with yeast. Thirty minutes prior to inoculation with yeast, glucoamylase was added to the corn mash. Erlenmyer flasks (500 ml size) were used with needle traps for the experiment. Three hundred (300 g) grams of the above prepared corn mash was weighed into each flask. Triplicate flasks for each yeast strain were prepared. Yeast (each type) was hydrated in water at 20 C for 1.5 to 2 hours then 7.5×10̂9 yeast (25×10̂6/gram of corn mash) was inoculated into each flask. The contents of the flasks were stirred and then placed into an incubator shaking at 120 rpm and with a temperature profile at 34 C for 24 hours, then changed to 31 C until harvest at 65 hours of fermentation time. Flasks were stirred after 24 and 48 hours of incubation. Samples were collected and centrifuged at 4100×g for seven minutes. The resulting supernatant was filtered through a 0.45 micron filter and then placed into an HPLC tube for analysis. The HPLC instrument was calibrated using the Ethanol Industry Standard (Midland Scientific, NE). Samples were run for 12.5 minutes on an HPX87H column and the data was printed and analyzed.

| | | Glycerol | Glycerol | Ethanol | Ethanol |
|---|---|---|---|---|---|
| Yeast Description | ploidy | (w/v %) | Std Dev | (w/v %) | Std Dev |
| a/α fps1::URA3 PGKp-GLT1 | diploid | 1.284 | 0.1 | 12.682 | 0.073 |
| gpd2::ura3 | | | | | |
| Mata fps1::URA3 PGKp-GLT1 | haploid | 1.337 | 0.006 | 13.481 | 0.056 |
| gpd2::ura3 PT₆₀-GPD1 | | | | | |
| (ATCC Accession No. | | | | | |
| PTA-10766) | | | | | |
| Commercial Yeast #1 | diploid | 1.743 | 0.006 | 13.115 | 0.056 |
| (BIOFERM XR) | | | | | |
| Commercial Yeast #2 | diploid | 1.754 | 0.009 | 13.114 | 0.036 |
| (ETHANOL RED ®) | | | | | |

Data for Glycerol and Ethanol are the average of triplicates
Std Dev = Standard deviation of the three results/yeast strain

### Example 4

### Ethanol production for Mata fps1::URA3 PGKp-GLT1 gpd2::ura3 PT₆₀-GPD1 as Compared to Another Haploid Yeast Strain S288C

Corn mash was prepared at ˜32.6% sugars with a 33.05% solids as measured by a microwave moisture analyzer. Standard preparation of the corn mash was completed using freshly ground corn and Alpha-amylase enzyme. The approximate Brix of the material was 27.0 and the starting pH of the corn mash was 4.78. A total of 450 ppm of Nitrogen (270 ppm from Ammonia and 180 ppm N from urea) along with 5 ppm of each of the following antibiotics, Allpen and Lactoside V were put into the corn mash prior to inoculation with yeast. Thirty minutes prior to inoculation with yeast, glucoamylase was added to the corn mash. Erlenmyer flasks (500 ml size) were used with needle traps for the experiment. Three hundred (300 g) grams of the above prepared corn mash was weighed into each flask. Triplicate flasks for each yeast strain were prepared. Yeast (each type) was hydrated in water at 20 C for 1.5 to 2 hours then 7.5×10̂9 yeast (25×10̂6/gram of corn mash) was inoculated into each flask. The contents of the flasks were stirred and then placed into an incubator shaking at 120 rpm and with a temperature profile at 34 C for 24 hours, then changed to 31 C. Samples were harvested at 24, 48, 65, 70 and 87 hours of fermentation time. Flasks were stirred during each sample collection time. Samples were collected and centrifuged at 4100×g for seven minutes. The resulting supernatant was filtered through a 0.45 micron filter and then placed into an HPLC tube for analysis. The HPLC instrument was calibrated using the Ethanol Industry Standard (Midland Scientific, NE). Samples were run for 12.5 minutes on an HPX87H column and the data was printed and analyzed.

| | Ethanol Production (w/v %) | | | | |
|---|---|---|---|---|---|
| Yeast Genotype (ploidy) | T = 24 | T = 48 | T = 65 | T = 70 | T = 87 |
| S288C (1N) | 7.431 | 11.013 | 11.424 | 11.546 | 11.516 |
| Mata fps1::URA3 PGKp- | 7.403 | 12.230 | 13.083 | 13.096 | 13.207 |
| GLT1 gpd2::ura3 PT₆₀- | | | | | |
| GPD1 (1N) | | | | | |
| (ATCC Accession No. | | | | | |
| PTA-10766) | | | | | |

Data for Ethanol are the average of triplicates

### Example 5

### Analytes of distillates from Mata fps1::URA3 PGKp-GLT1 gpd2::ura3 PT₆₀-GPD1 as Compared to Other Commercially Available Yeast Cells and a/αfps1::URA3 PGKp-GLT1 gpd2::ura3

Approximately 250 g corn mash (after fermentation) and 100 g water were added into a 1000 ml round bottom distillation flask. The flask was placed in a heating mantle and connected to a simple distillation apparatus consisting of a distillation head with ˜10 plates and a cold water condenser. The flask was heated using the heating mantle at high setting. Exactly 100 ml condensate distill was collected into a 100 ml volumetric receiving flask. The approximate proof was checked by hydrometry. The distillate should be about 60 proof (+/−3 proof). The distillate was analyzed for alcohol by HPLC to obtain % Ethanol w/v. The distillate was then analyzed for higher alcohols (fusel oils) by Gas Chromatography. One (1) ml distillate plus 0.1 ml internal standard was pipeted into a 2 ml sample vial and placed in the autosampler tray of a Agilent 7980A GC system. The GC method's calibration table was first verified with a prepared fusel oil standard of known concentrations. Calculations based on area for each compound in the sample were then calculated to determine sample value (e.g., area under curve of sample). The results of the analysis were then normalized to 100 proof for comparison purposes (each distillate does not have the exactly same proof).

| | | Mata fps1::URA3 | | |
|---|---|---|---|---|
| | | PGKp-GLT1 | | |
| | | gpd2::ura3 | | |
| | a/α fps1::URA3 | PT₆₀-GPD1 | | |
| | PGKp-GLT1 | (ATCC Accession | Commercial #1 | Commercial #2 |
| | gpd2::ura3 | No. PTA-10766) | (BIOFERM XR) | (ETHANOL RED ®) |
| | Conc. mg/L | Conc. mg/L | Conc. mg/L | Conc. mg/L |
| Analyte | at 100 °P | at 100 °P | at 100 °P | at 100 °P |
| Acetaldehyde | 115.0 | 128.0 | 58.1 | 55.0 |
| Methanol | 29.0 | 15.0 | 27.8 | 26.5 |
| Ethyl acetate | 40.8 | 36.5 | 45.5 | 44.7 |
| n-Propanol | 204.2 | 144.8 | 139.7 | 144.9 |
| sec-Butanol | 0.0 | 0.0 | 0.0 | 0.0 |
| iso-Butanol | 407.6 | 182.7 | 275.7 | 248.7 |
| n-Butanol | 10.2 | 0.0 | 0.0 | 0.0 |
| Acetic acid | 94.3 | 101.1 | 128.8 | 123.4 |
| a-Amyl alcohol | 407.0 | 202.2 | 268.5 | 235.1 |
| iso-Amyl | 843.3 | 496.5 | 776.6 | 684.1 |
| alcohol | | | | |

100 °P = 100 proof alcohol

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A genetically-altered haploid yeast cell characterized by:
a. reduced activity or expression of FPS1;
b. reduced activity or expression of glycerol-3-phosphate dehydrogenase-1 (GPD1);
c. reduced activity or expression of glycerol-3-phosphate dehydrogenase-2 (GPD2); and
d. increased expression of glutamate synthase (GLT1),
wherein the reduced activity or expression, and the increased expression, is relative to expression or activity in a wildtype yeast strain.

2. The haploid yeast cell of claim 1, having reduced expression of FPS1, GPD1, and GPD2.

3. The haploid yeast cell of claim 1, comprising a deletion in the coding region of FPS1, thereby preventing or reducing expression of an active FPS1 protein.

4. The haploid yeast cell of claim 1, comprising a deletion in the coding region of GPD2, thereby preventing or reducing expression of an active GPD2 protein.

5. The haploid yeast cell of claim 1, comprising a deletion in the coding region of GPD1, thereby preventing or reducing expression of an active GPD1 protein.

6. The haploid yeast cell of claim 1, comprising a deletion in the promoter region of FPS1, thereby preventing or reducing expression of an active FPS1 protein.

7. The haploid yeast cell of claim 1, comprising a deletion in the promoter region of GPD2, thereby preventing or reducing expression of an active GPD2 protein.

8. The haploid yeast cell of claim 1, comprising a deletion in the promoter region of GPD1, thereby preventing or reducing expression of an active GPD1 protein.

9. The haploid yeast cell of claim 1, wherein GLT1 expression is increased due to a heterologous promoter operably linked to a GLT1 coding sequence, wherein the heterologous promoter causes increased expression of GLT1.

10. The haploid yeast cell of claim 9, wherein the heterologous promoter comprises a yeast phosphoglycerate kinase (PGK) promoter.

11. The haploid yeast cell of claim 1, comprising
a deletion in the coding region of FPS1, thereby preventing or reducing expression of an active FPS1 protein;
a deletion in the coding region of GPD2, thereby preventing or reducing expression of an active GPD2 protein;
a deletion in the promoter region of GPD1, thereby preventing or reducing expression of an active GPD1 protein; and
a heterologous promoter operably linked to a GLT1 coding sequence, wherein the heterologous promoter causes increased expression of GLT1.

12. The haploid yeast cell of claim 11, wherein the yeast cell is genetically altered from Saccharomyces cerevisiae, S. bayanus, S. pastorianus, Pichia stipitis, or Candida shehatae.

13. A genetically-altered haploid yeast cell as deposited as ATCC Accession No. PTA-10766.

14. A method of generating ethanol from sugar, the method comprising, contacting the yeast cell of claim 1 to a solution comprising sugar under conditions such that the yeast cell converts the sugar to ethanol.

15. A system comprising a fermentation bioreactor and a yeast cell of claim 1.


SEQUENCE LISTING (26 sequences)

SEQ ID NO: 1 - DNA, 551 bp, Artificial Sequence
  synthetic Mata fps1::URA3 PGKp-GLT1 gpd2::ura3 glycerol-3-phosphate dehydrogenase-1 (GPD1) promoter (PT60 GPD1)
ccacttctacttctacatcggaaaaacattccattcacatatcgtctttggcctatcttgttttgtccttggtagatcaggtcagtacaaacgcaacacgaaagaacaaaaaaagaagaaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctcccctcccttttctttttccagttttccctattttgtccctttttccgcacaacaagtatcagaatgggttcatcaaatctatccaacctaattcgcacgtagactggcttggtattggcagtttcgcagttatatatatactaccatgagtgaaactgttacgttaccttaaattctttctccctttaattttcttttatcttactctcctacataagacatcaaatg

SEQ ID NO: 2 - DNA, 611 bp, Saccharomyces cerevisiae
  promoter (1)...(611): wild type glycerol-3-phosphate dehydrogenase-1 (GPD1) promoter
ccacttctacttctacatcggaaaaacattccattcacatatcgtctttggcctatcttgttttgtccttggtagatcaggtcagtacaaacgcaacacgaaagaacaaaaaaagaagaaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctcccctcccttttctttttccagttttccctattttgtccctttttccgcacaacaagtatcagaatgggttcatcaaatctatccaacctaattcgcacgtagactggcttggtattggcagtttcgcagttatatatatactaccatgagtgaaactgttacgttaccttaaattctttctccctttaattttcttttatcttactctcctacataagacatcaagaaacaattgtatattgtacaccccccccctccacaaacacaaatattgataatataaagatg

SEQ ID NO: 3 - DNA, 32 bp, Artificial Sequence
  synthetic PCR amplification primer Rep1-U
gggcccggatccgagcagcataaacgactgct

SEQ ID NO: 4 - DNA, 32 bp, Artificial Sequence
  synthetic PCR amplification primer Rep1-D
gggccctctagaacgctcaatgttgttcatga

SEQ ID NO: 5 - DNA, 32 bp, Artificial Sequence
  synthetic PCR amplification primer URA3-U
gggccctctagagtagtctagtacctcctgtg

SEQ ID NO: 6 - DNA, 32 bp, Artificial Sequence
  synthetic PCR amplification primer URA3-D
gggcccgtcgacgaaaagtgccacctgacgtc

SEQ ID NO: 7 - DNA, 32 bp, Artificial Sequence
  synthetic PCR amplification primer Rep2-U
gggcccgtcgacgagcagcataaacgactgct

SEQ ID NO: 8 - DNA, 32 bp, Artificial Sequence
  synthetic PCR amplification primer Rep2-D
gggcccctgcagacgctcaatgttgttcatga

SEQ ID NO: 9 - DNA, 59 bp, Artificial Sequence
  synthetic PCR amplification primer KFPS1-U
tcaacaaagtataacgcctattgtcccaataagcgtcggtacgacgttgtaaaacgacg

SEQ ID NO: 10 - DNA, 59 bp, Artificial Sequence
  synthetic PCR amplification primer KFPS1-D
catcatgtatagtaggtgaccaggctgagttcatgtcaacggaaacagctatgaccatg

SEQ ID NO: 11 - DNA, 59 bp, Artificial Sequence
  synthetic PCR amplification primer KGPD2-U
ctctttccctttccttttccttcgctccccttccttatcaacgacgttgtaaaacgacg

SEQ ID NO: 12 - DNA, 59 bp, Artificial Sequence
  synthetic PCR amplification primer KGPD2-D
gcaacaggaaagatcagagggggagggggggggagagtgtggaaacagctatgaccatg

SEQ ID NO: 13 - DNA, 32 bp, Artificial Sequence
  synthetic PCR amplification primer GLT1-U
gggcccgtcgacatgccagtgttgaaatcaga

SEQ ID NO: 14 - DNA, 32 bp, Artificial Sequence
  synthetic PCR amplification primer GLT1-D
gggcccctgcagttttagtatcgaccatttca

SEQ ID NO: 15 - DNA, 32 bp, Artificial Sequence
  synthetic PCR amplification primer GLT1prom-U
gggcccggtacctttctgagcactgtcaggag

SEQ ID NO: 16 - DNA, 32 bp, Artificial Sequence
  synthetic PCR amplification primer GLT1prom-D
gggcccggatcctgatttcaacactggcatgc

SEQ ID NO: 17 - DNA, 33 bp, Artificial Sequence
  synthetic PCR amplification primer PGK1prom-U
gggcccggatccaggcatttgcaagaattactc

SEQ ID NO: 18 - DNA, 38 bp, Artificial Sequence
  synthetic PCR amplification primer PGK1prom-D
gggcccgtcgactgttttatatttgttgtaaaaagtag

SEQ ID NO: 19 - DNA, 31 bp, Artificial Sequence
  synthetic PCR amplification primer pGPD1-U
gggcccgaattccacttctacttctacatcg

SEQ ID NO: 20 - DNA, 33 bp, Artificial Sequence
  synthetic PCR amplification primer pGPD1-D
gggcccggatccttgatgtcttatgtaggagag

SEQ ID NO: 21 - DNA, 32 bp, Artificial Sequence
  synthetic PCR amplification primer GPD1-U
gggcccggatccatgtctgctgctgctgatag

SEQ ID NO: 22 - DNA, 32 bp, Artificial Sequence
  synthetic PCR amplification primer GPD1-D
gggcccaagctttgagtggtgttgtaaccacc

SEQ ID NO: 23 - DNA, 632 bp, Artificial Sequence
  synthetic PT-60-GPD1 locus
ccacttctacttctacatcggaaaaacattccattcacatatcgtctttggcctatcttgttttgtccttggtagatcaggtcagtacaaacgcaacacgaaagaacaaaaaaagaagaaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctcccctcccttttctttttccagttttccctattttgtccctttttccgcacaacaagtatcagaatgggttcatcaaatctatccaacctaattcgcacgtagactggcttggtattggcagtttcgcagttatatatatactaccatgagtgaaactgttacgttaccttaaattctttctccctttaattttcttttatcttactctcctacataagacatcaaggatccatgtctgctgctgctgatagattaaacttaacttccggccacttgaatgctggtagaaagagaagttcctcttctgtt

SEQ ID NO: 24 - DNA, 1147 bp, Artificial Sequence
  synthetic PGKp-GLT1 locus
cgtctccacatcataggaagataggaaattgctatctcagtcctatactacgcagacggatactctcagttgctctttcttccccttcttttagctcattgaggtagtgattaacgtttaacttatttatttatttttctgcttcagtttttttttatttatttttgtctttctactctctcttttttcttaatctatttgccatttatttattttgaagaactagaaaaagaattagaaaagaaagcatgccagtgttgaaatcaggatccaggcatttgcaagaattactcgtgagtaaggaaagagtgaggactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttcccacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacagtcgacatgccagtgttgaaatcagacaatttcgatccattggaagaagcttacgaaggtgggacaattcaaaactataacgatgaacaccatcttcataaatcttgggcaaatgtgattccggacaaacgaggactttacgaccctgattatga

SEQ ID NO: 25 - DNA, 1593 bp, Artificial Sequence
  synthetic gpd2::ura3 locus
ttttttttatatattaatttttaagtttatgtattttggtagattcaattctctttccctttccttttccttcgctccccttccttatcaatgctaagagatagtgatgatatttcataaataatgtaattctatatatgttaattaccttttttgcgaggcatatttatggtgaagaataagttttgaccatcaaagaaggttaatgtggctgtggtttcagggtccataaagcttttcaattcatcatttttttttattcttttttttgattccggtttccttgaaatttttttgattcggaaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattacagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgaagtggtctctacaggatctgacattattattgttgaaagagggctatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaattggaaagaaaaagcttcatggcctttataaaaaggaactatccaatacctcacactctccccccccctccccctctgatctttcctgttgcctctttttcccccaaccaatttatcattatacacaagttctacaactactactagtaatattactacagttattataattttctattctctttttctttaagaatctatcattaacgttaat

SEQ ID NO: 26 - DNA, 2080 bp, Artificial Sequence
  synthetic fps1::URA locus
ttttgcggaaagataaaacaagatatattgcactttttccaccaagaaaaacaggaagtggattaaaaaatcaacaaagtataacgcctattgtcccaataagcgtcggttgttcttctttattattttaccaagtacgctcgagggtacattctaatgcattaaaagacatgctaagagatagtgatgatatttcataaataatgtaattctatatatgttaattaccttttttgcgaggcatatttatggtgaagaataagttttgaccatcaaagaaggttaatgtggctgtggtttcagggtccataaagcttttcaattcatcattttttttttattcttttttttgattccggtttccttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgccgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcaattattacccgggaatctcggtcgtaatgatttctataatgacgaaaaaaaaaaaattggaaagaaaaagcttcatggcctttataaaaaggaacatccaataccttgagaaaacagacaagaaaaagaaacaaataatatagactgatagaaaaaaataccgcttactaccgccggtataatatatatatatatatttacatagatgattgcatagtgttttaaaaagctttcctaggttaagctatgaatcttcataacctaaccaactaaatatgaaaatactgacccatcgtcttaagtaagttgacatgaactcagcctggtcacctactatacatgatgtatcgcatggatggaaagaataccaaacgctaccttccaggttaatgatagtatccaaacctagttggaatttgccttgaacatcaagcagcgattcgatatcagttgggagcatcaatttggtcattggaataccatctatgcttttctcctcccatattctcaaaagtagtaagggctcgttatatacttttgaatatgtaagatataattctatatgatttagtaatttattttctatacgctcagtatttttctgcagttgtcgagtaggtattaaacgcagaagaagtccatccttctcatcattcaaatgaacatcttggcaaa